SEQUENCE LISTING (41 sequences)

SEQ ID NO: 1 - protein, 11 aa, H. lori
  VARIANT (4)...(4): Any amino acid
MDLXVLGINTA

SEQ ID NO: 2 - protein, 13 aa, H. pylori
MRVPKKGFAILSK

SEQ ID NO: 3 - protein, 13 aa, H. pylori
MRVPSKGFAILSK

SEQ ID NO: 4 - protein, 11 aa, H. pylori
  VARIANT (1)...(2): Any amino acid
XXGKAPDFKPA

SEQ ID NO: 5 - protein, 8 aa, H. pylori
MLKIKLEI

SEQ ID NO: 6 - protein, 8 aa, H. pylori
MLKIKLSI

SEQ ID NO: 7 - protein, 8 aa, H. pylori
MLKIVLEI

SEQ ID NO: 8 - protein, 8 aa, H. pylori
MLKIVLSI

SEQ ID NO: 9 - protein, 8 aa, H. pylori
MLKISLEI

SEQ ID NO: 10 - protein, 8 aa, H. pylori
MLKISLSI

SEQ ID NO: 11 - protein, 8 aa, H. pylori
MLKEKLEI

SEQ ID NO: 12 - protein, 8 aa, H. pylori
MLKEKLSI

SEQ ID NO: 13 - protein, 8 aa, H. pylori
MLKEVLEI

SEQ ID NO: 14 - protein, 8 aa, H. pylori
MLKEVLSI

SEQ ID NO: 15 - protein, 8 aa, H. pylori
MLKESLEI

SEQ ID NO: 16 - protein, 8 aa, H. pylori
MLKESLSI

SEQ ID NO: 17 - protein, 8 aa, H. pylori
MLVIKLEI

SEQ ID NO: 18 - protein, 8 aa, H. pylori
MLVIKLSI

SEQ ID NO: 19 - protein, 8 aa, H. pylori
MLVIVLEI

SEQ ID NO: 20 - protein, 8 aa, H. pylori
MLVIVLSI

SEQ ID NO: 21 - protein, 8 aa, H. pylori
MLVISLEI

SEQ ID NO: 22 - protein, 8 aa, H. pylori
MLVISLSI

SEQ ID NO: 23 - protein, 8 aa, H. pylori
MLVEKLEI

SEQ ID NO: 24 - protein, 8 aa, H. pylori
MLVEKLSI

SEQ ID NO: 25 - protein, 8 aa, H. pylori
MLVEVLEI

SEQ ID NO: 26 - protein, 8 aa, H. pylori
MLVEVLSI

SEQ ID NO: 27 - protein, 8 aa, H. pylori
MLVESLEI

SEQ ID NO: 28 - protein, 8 aa, H. pylori
MLVESLSI

SEQ ID NO: 29 - protein, 8 aa, H. pylori
  VARIANT (5)...(5): Any amino acid
MLTIXLEV

SEQ ID NO: 30 - protein, 8 aa, H. pylori
  VARIANT (5)...(5): Any amino acid
MLTIXLEE

SEQ ID NO: 31 - protein, 8 aa, H. pylori
  VARIANT (5)...(5): Any amino acid
KLTIXLEV

SEQ ID NO: 32 - protein, 8 aa, H. pylori
  VARIANT (5)...(5): Any amino acid
KLTIXLEE

SEQ ID NO: 33 - protein, 8 aa, H. pylori
MYIPYVIE

SEQ ID NO: 34 - protein, 8 aa, H. pylori
MNLDCLQV

SEQ ID NO: 35 - protein, 8 aa, H. pylori
MNLDSLQV

SEQ ID NO: 36 - protein, 20 aa, H. pylori
GKIGIFFGTDSGNAEAIAEK

SEQ ID NO: 37 - protein, 15 aa, H. pylori
  VARIANT (14)...(14): Any amino acid
MLVTKLAPDFLAPXV

SEQ ID NO: 38 - protein, 20 aa, H. pulori
MFTLRELPFAKDSNGDFLSP

SEQ ID NO: 39 - protein, 10 aa, H. pylori
MVTLINNEDD

SEQ ID NO: 40 - protein, 8 aa, H. pylori
AKEIKFSD

SEQ ID NO: 41 - protein, 8 aa, H. pylori
MKKISRKE

(19) United States
(12) Patent Application Publication
(10) Pub. No.: US 2002/0187161 A1
(43) Pub. Date: Dec. 12, 2002

(54) H. pylori antigens

(76) Inventors: Allan William Cripps, Farrer (AU); Robert Llewellyn Clancy, Newcastle (AU); Lois McShane, Hawks Nest (AU); Christopher John Smith, Rhauallt (GB); David Robert Tyreman, Llandullas (GB); Bow Ho, Singapore (SG)

Correspondence Address: BAKER & BOTTS, 30 ROCKEFELLER PLAZA, NEW YORK, NY, 10112

(21) Appl. No.: 10/047,881

(22) Filed: Jan. 14, 2002

Related U.S. Application Data
(63) Continuation of application No. 09/358,423, filed on Jul. 22, 1999.
Application No. 09/358,423 is a continuation-in-part of application No. PCT/GB98/00220, filed on Jan. 26, 1998.

(30) Foreign Application Priority Data
Jan. 24, 1997 (GB) 9701487.2
May 22, 1997 (GB) 9710629.8

(51) Int. Cl. A61K 39/02; C07K 7/06; C07K 7/08

(52) U.S. Cl. 424/190.1; 530/326; 530/327; 530/328

(57) ABSTRACT

Novel antigens from H. pylori are provided. Their use in diagnosing H. pylori infection is also disclosed, including methods for said diagnosis, and kits for use in such a method. In addition, novel antigenic fragments of the antigens are provided, as well as vaccines comprising either at least one of the antigens or one or more antigenic fragments.

The present invention relates to novel antigens of Helicobacter pylori, or antigenic fragments thereof, the use of the antigen or fragments thereof in detecting Helicobacter pylori and kits comprising them, as well as vaccines comprising the antigens or fragments thereof and a method for isolation of the antigen.

Gut infections in mammals, and in particular humans, stimulate an immune response in mucous secretions, such as saliva, through activation of the common mucosal immune system. This response often initially parallels an antibody response in serum although it is generally characterised by the presence of IgA antibodies. However, the immune response in secretion, including saliva, rapidly diminishes following elimination of the antigen (eg bacteria or virus) from the body. Accordingly, the presence of antibody in mucous secretions reflects current, ie contemporary, infection. In the case of a microbial infection, for example, antibodies in mucous secretions, hereinafter referred to as secretions antibodies, reflect the current status of colonisation of the microbe, such as in the gut, and thus is a useful monitor of contemporary infection. Serum antibody, on the other hand, persists for some time after the microbe is eliminated from the body. A positive serum antibody test, therefore, reflects both past and present exposure to antigen which is less helpful to the clinician. A positive secretious antibody test, on the other hand, indicates present or contemporary infection by the microbe.

The diagnosis of H. pylori infection can be made by microscopy, microbiological culture or urease detection in gastric mucosal biopsies, urea breath test or by the presence of specific antibodies in serum ELISAs. It might be predicted that H. pylori infection, being an infection of the gastric mucosa, would elicit an IgA antibody response in gastric secretion. However, it has been discovered that H. pylori-specific antibody in mucous secretions is of the IgG class and not IgA as might have been expected. Little IgA antibody, if any, is detected. Accordingly, AU-A-9067676 is directed to the detection of IgG in mucous secretion specific to H. pylori antigen and thereby provides a means of monitoring current, ie contemporary, infection by that microorganism in mammals. The corresponding academic publication is Witt et al, Frontiers in Mucosal is Immunology 1 693-696 (1991).

The presence of IgG antibodies in the saliva of Helicobacter pylori positive patients has received some attention in the proceedings of the Annual Meetings of the American Gastroenterological Association. After the disclosure by Czinn et al of the presence of such antibodies in the 1989 proceedings, Larsen et al concluded in the May 1991 proceedings that salivary IgG levels are a practical, non-invasive marker of therapeutic response during a course of antibiotic therapy. In the April 1992 proceedings, Landes et al confirmed earlier observations and observed that measurement of salivary IgG to Helicobacter pylori is a simple, non-invasive test for detecting H. pylori positive patients, especially in widespread or paediatric populations where other tests are not practical.

WO-A-9322682 discloses a convenient and reliable in vitro test for H. pylori. This test utilises an antigen preparation in a reaction with IgG antibody in a mucous secretion from a mammal being tested.

WO-A-9625430 discloses a novel antigen from H.pylori which can be used in diagnostic tests for the identification of H.pylori infection.

There is a continuing need to identify, isolate and thus provide novel antigens from H. pylori which can be used in diagnostic tests. These antigens should be specific, reliably purifiable, and should be characterised by good specificity and the lack of false positive results when used in such tests. In addition they may also form the basis of a vaccine useful either for the treatment or prophylaxis of H.pylori infection.

Thus, in a first aspect, the present invention provides a protein being an H. pylori antigen and having a molecular weight in the range of about 43 kDa to about 53 kDa, as determined under denaturing and reducing conditions.

In one preferred embodiment the antigenic protein has a molecular weight of about 43 kDa and has, at its amino terminal end, the following amino acid sequence:

M D L ? V L G I N T A
Met-Asp-Leu- ? -Val-Leu-Gly-Ile-Asn-Thr-Ala.

In a second preferred embodiment the antigenic protein has a molecular weight of about 43 kDa and has, at its amino terminal end, the following amino acid sequence:

M R V P K(S) K G F A I L S K

In a third preferred embodiment of this aspect of the invention the antigenic protein has a molecular weight of about 53 kDa and has, at its amino terminal end, the following amino acid sequence:

? ? G K A P D F K P A
? -? -Gly-Lys-Ala-Pro-Asp-Phe-Lys-Pro-Ala

In a second aspect the present invention provides a protein being an H. pylori antigen and having the following characteristics:

i) a molecular weight of about 54 kDa, as determined under denaturing and reducing conditions and the following N-terminal amino acid sequence:

M L K (V) I (E) K (V or S) L E (S) I;

ii) a molecular weight of about 370 kDa, as determined under native (non-denaturing) conditions and the following N-terminal amino acid sequence:

M (K) L T I - L E V (E);

iii) a molecular weight of about 140 kDa, as determined under native (non-denaturing) conditions and the following N-terminal amino acid sequence:

M Y I P Y V I E;

iv) a molecular weight of about 90 kDa, as determined under native (non-denaturing) conditions and the following N-terminal amino acid sequence:

M N L D C (S) L Q V;

v) a molecular weight of about 15.9 to 16.9 kDa, as determined under denaturing-and reducing conditions, and the following N-terminal amino acid sequence:

G K I G I F F G T D S G N A E A I A E K;

vi) a molecular weight of about 344 kDa, as determined under native (non-denaturing) conditions, and the following N-terminal amion acid sequence;

M I V T K L A P D F L A P ? V; or

vii) a superoxide dismutase enzyme having the following N-terminal amino acid sequence:

M F T L R E L P F A K D S N G D F L S P

In the above sequences bracketed amino acids represent alternatives to the preceding one.

Parts or fragments of any of the whole proteins described herein may themselves be antigenic and thus, in a third aspect, the present invention provides an antigenic fragment of a protein of the invention. In particular, the invention provides antigenic fragments having the following sequence:

```
  M D L ? V L G I N T A
Met-Asp-Leu- ? -Val-Leu-Gly-Ile-Asn-Thr-Ala;
            ? ? G K A P D F K P A
            ? - ? -Gly-Lys-Ala-Pro-Asp-Phe-Lys-Pro-Ala.
M L K(V) I(E) K(V or S) L E(S) I;
M R V P K(S) K G F A I L S K;
M(K) L T I - L E V(E);
M Y I P Y V I E;
M N L D C(S) L Q V;
G K I G I F F G T D S G N A E A I A E K;
M L V T K L A P D F L A P ? V; or
M F T L R E L P F A K D S N G D F L S P
```

wherein letters in brackets in the above sequences denote alternative amino acids to the preceding one.

The molecular weight of the antigens described herein are of necessity approximate figures, because of the limitations of molecular weight determination procedures. The molecular weights specifically referred to have been obtained using either native (non-denaturing) or denaturing conditions. Those skilled in the art will be aware that slightly different results can be obtained in different hands or even on differrent occasions in the same hands, and so the approximate molecular weight figures quoted in this specification should be read as ±5% or even ±10%.

The skilled man will appreciate that some variation in the sequence of fragments will be possible, while still retaining antigenic properties. Methods well known to the skilled person can be used to test fragments and/or variants thereof for antigenicity. Such variants also form part of the invention.

The antigenic proteins or fragments thereof, of the present invention can be provided alone, as a purified or isolated preparation, or as part of a mixture with other H. pylori antigenic proteins.

In a fourth aspect, therefore, the invention provides an antigen composition comprising one or more proteins of the invention and/or one or more antigenic fragments thereof. Such a composition can be used for the detection and/or diagnosis of H. pylori . In one embodiment the composition comprises one or more additional H. pylori antigens or fragments thereof.

In a fifth aspect, the present invention provides a method of detecting and/or diagnosing H. pylori which comprises:

(a) bringing into contact an antigenic protein, or antigenic fragment thereof, or an antigen composition of the invention with a sample to be tested; and

(b) detecting the presence of antibodies to H. pylori.

In particular, the proteins, antigenic fragments thereof or antigen composition of the invention can be used to detect IgG antibodies. Suitably, the sample to be tested will be a biological sample, e.g. a sample of blood or saliva. An example of a suitable method for detection of H.pylori using a sample of a mucous secretion is that described in WO-A-9322682.

In a sixth aspect, the invention provides the use of an antigenic protein, antigenic fragment thereof or antigenic composition of the present invention in detecting and/or diagnosing H. pylori. Preferably, the detecting and/or diagnosing is carried out in vitro.

The antigenic protein, antigenic fragment thereof or antigen composition of the invention can be provided as part of a kit for use in in vitro detection and/or diagnosis of H.pylori. Thus, in a seventh aspect, the present invention provides a kit for use in the detection and/or diagnosis of H.pylori comprising an antigenic protein, antigenic fragment thereof or antigen composition of the invention.

In addition, antigenic protein or antigenic fragment thereof of the invention can be used to induce an immune response against H. pylori. Thus, in a further aspect, the present invention provides the use of an antigen of the invention, a fragment thereof or an antigenic composition of the invention in medicine.

In yet a further aspect the present invention provides a composition capable of eliciting an immune response in a subject which comprises one or more proteins and/or one or more antigenic fragments thereof of the invention. Suitably, the composition will be a vaccine composition, optionally comprising one or other suitable adjuvants. Such a vaccine composition may be either a prophylactic or therapeutic vaccine composition.

The vaccine compositions of the invention can include one or more adjuvants. Examples of adjuvants well known in the art include inorganic gels such as aluminium hydroxide or water-in-oil emulsions such as incomplete Freund's adjuvant. Other useful adjuvants will be well known to the skilled man.

In yet further aspects, the present invention provides:

(a) the use of a protein or one or more antigenic fragments thereof of the invention in the preparation of an immunogenic composition, preferably a vaccine;

(b) the use of such an immunogenic composition in inducing an immune response in a subject; and

(c) a method for the treatment or prophylaxis of H. pylori infection in a subject, which comprises the step of administering to the subject an effective amount of a protein, at least one antigenic fragment thereof or an antigen composition of the invention, preferably as a vaccine.

Preferred features of each aspect of the invention are as for each other aspect mutatis mutandis.

The invention will now be described with reference to the following example which should not be construed as limiting the invention in any way.

The examples refer to the figures in which:

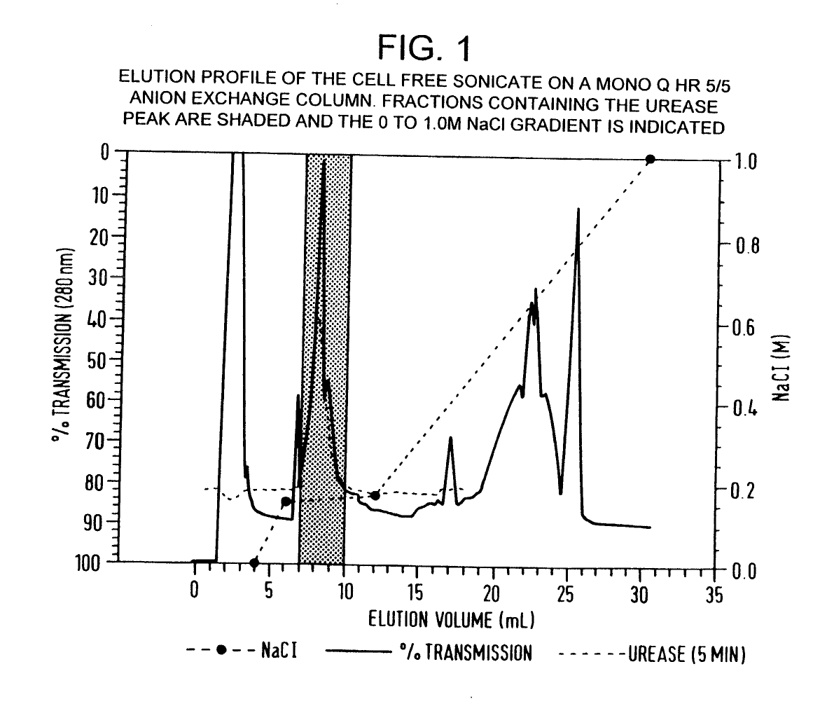

FIG. 1: shows the elution profile of the cell free sonicate on a mono Q HR 5/5 anion exchange column. Fractions which contain urease are indicated by the shaded area. The 0 to 1.0 M NaCl gradient is indicated;

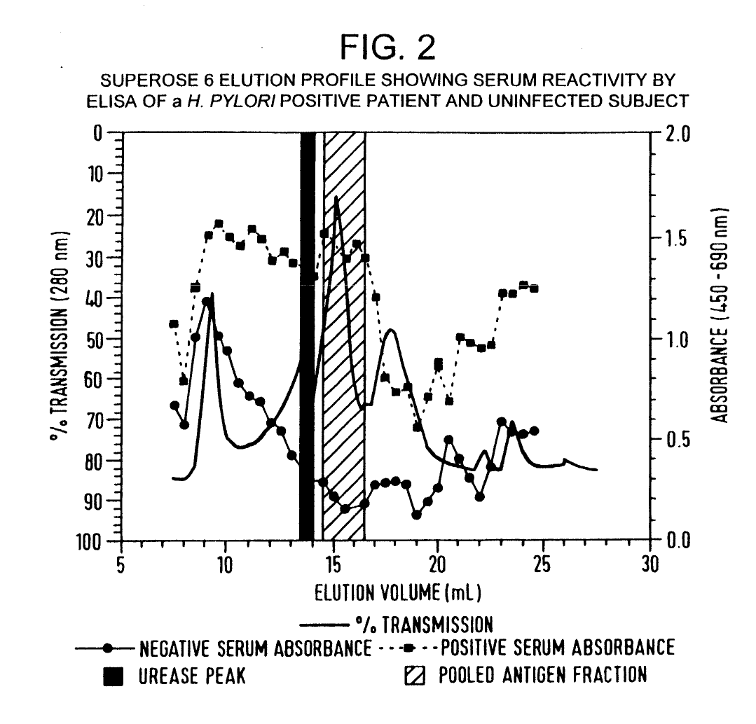

FIG. 2: shows a Superose 6 elution profile showing serum reactivity by ELISA of a H. pylori positive patient and an uninfected subject;

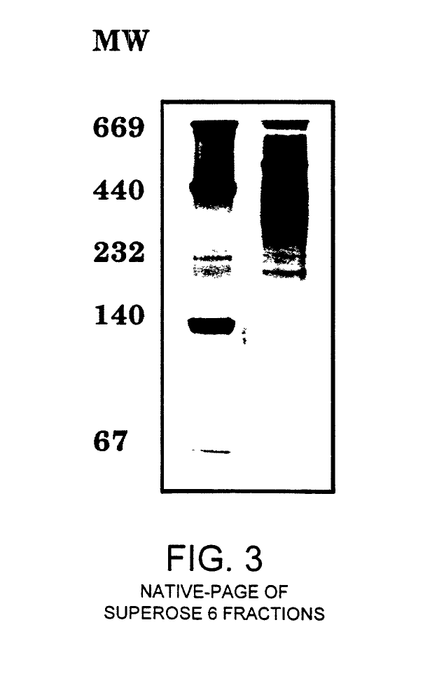

FIG. 3: shows native PAGE 8-25% gradient of the Superose 6 reactive fraction of the 2 strains of H. pylori studied;

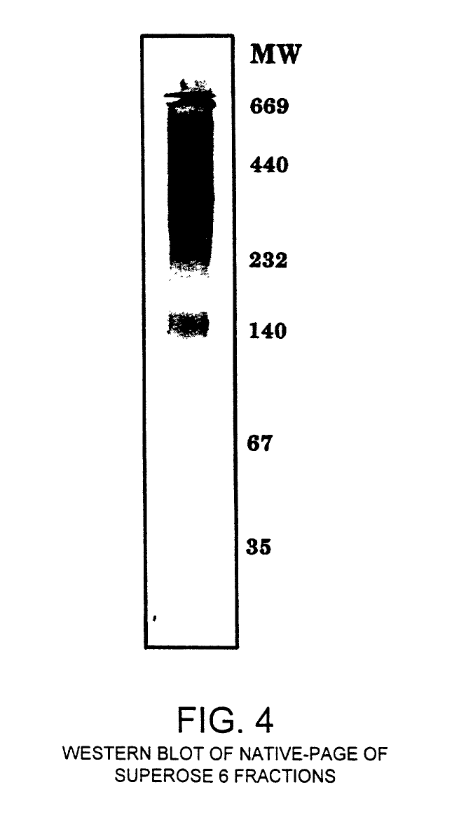

FIG. 4: shows a Western blot of Native PAGE 8-25% gradient of the Superose 6 reactive fraction;

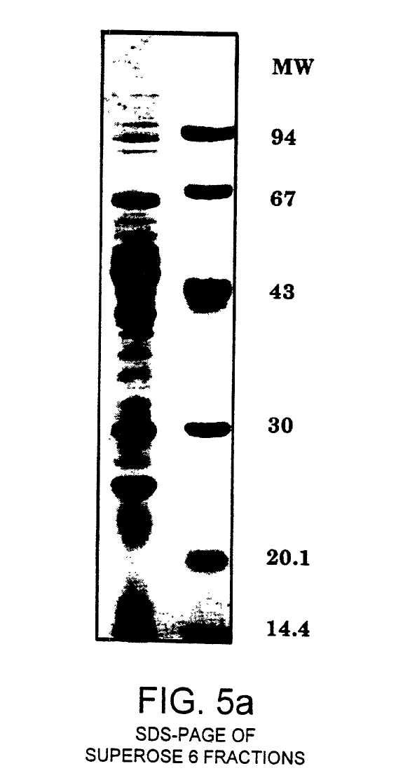

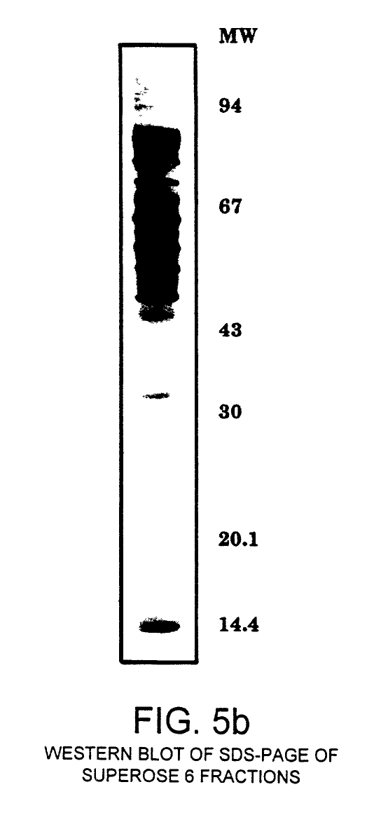

FIG. 5: shows (a) SDS-PAGE 8-25% gradient of the superose 6 reactive fraction and (b) Western blot of (a); and

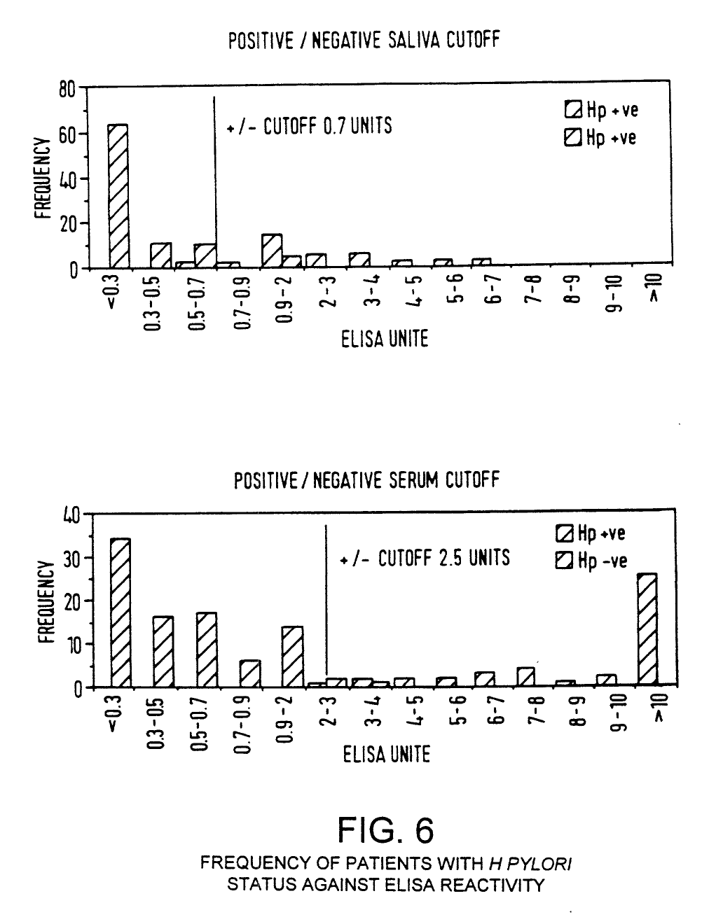

FIG. 6: shows frequency of patients with known H. pylori status against ELISA reactivity.

### EXAMPLE 1

(i) Bacterial Culture

Two strains of H.pylori were used, NCTC11637 and a wild type strain designated “traub” which was isolated from a patient with gastric ulceration in 1989 (Australian Institute of Mucosal Immunology).

The same proteins have been isolated from both strains. Each strain was cultured, and the proteins extracted in the same manner.

Bacteria were grown on Chocolate agar (Oxoid No 2 Block Agar Base-CM271-containing 5% defibrinated horse blood) in a water jacketed incubator at 37° C. with a micro-aerophilic atmosphere consisting of 10% CO₂ 6% O₂ and 84 N₂.

(ii) Sonication

After 96 hours culture plates were harvested by scraping colonies into a collection tube containing PBS (Trace MultiCel™Code 50-201-PA). Cells were washed by centrifugation (10,000 g, 5 minutes, RT) and resuspended in fresh buffer. This washing step was repeated once and cells were finally resuspended in 0.1 M Tris-HCl pH 8.2 prior to disruption by sonication. Sonication was carried out at 6μ (MSE Soniprep 150 Ultrasonic Disintegrator) in a cooled sonication tube seated in an ice-bath using a 9.5 mm probe. Sonication for a 1 ml aliquot consisted of 5 cycles each divided into 30 seconds sonication and 60 seconds rest giving a total sonication time of 7.5 minutes. After sonication cell debris was removed by centrifugation (12,000 g, 10 minutes, RT) and the suspension filtered initially through a 0.45 μm filter then through a 0.2 μm filter to produce a cell free suspension of proteins.

(iii) Protein Determination

Total protein concentration was determined using the BioRad Coomassie Blue Protein Determination kit.

(iv) Chromatography

(a) Ion Exchange

The cell free suspension was fractionated by application of the sample, 10-15 mg of protein in 500 μl of Tris-HCl buffer, to a Mono Q column (Pharmacia Biotech Ltd, HR 5/5) connected to an FPLC system. Elution was achieved with a gradient consisting of 0-1 M NaCl in 0.1 M tris-HCL buffer.

Protein elution was monitored at 280 nm and all the eluted material was collected in 0.5 ml fractions. The conductivity of the buffer was monitored throughout the procedure to ensure gradient accuracy.

Individual fractions were tested for urease activity by measuring their ability to utilise urea as a substrate. Briefly, using a micotitre plate, rows of wells were alternately filled with 90 μl of a solution consisting of either 3 mM disodium hydrogen phosphate plus 1.5% w/v urea and 4 μg/100 ml phenol red (substrate solution) or a similar solution excluding urea (buffer blank). Samples (10 μl) of each fraction were then added to the wells in pairs, one sample to the substarte solution and one sample to the buffer blank. immediately the plates were sealed. After 2.5 minutes the absorbance of each well was measured at 540 nm. The control values (buffer blank) were subtracted from the test value (substrate solution) for each pair of wells and the resultant values plotted against the fraction number.

(b) Gel Filtration Chromatography

Those Mono Q fractions shown to contain urease activity were combined to give three pools. Each pool was tested for antigenic activity. The first pool was shown to contain antigen and this pool was concentrated to give a total protein content of approximately 30-50 mg/ml. Aliquots (200 μl) of pool 1 were subjected to gel filtration chromatography on a Superose 6 column (Pharmacia). Elution was achieved using Tris-HCL, 0.1 M, pH 7.2 as the elution buffer. Fractions (0.5 ml) were collected. Elution was monitored by measuring the optical density of the eluate at 280 nm during the runs and subsequently by determining the urease activity and protein content.

(c) Selection of reactive fractions

Fractions were tested for antigen by diluting a sample 1 in 10 with Tris buffered saline containing 1 M NaCl and using these diluted samples to coat ELISA microtitre plate wells (Nunc Maxisorb), 100 μl per well. Wells were allowed to stand for 3 h then washed with 100 mM phosphate buffer with 0.15 M NaCl. Coated plates were then screened using a group of serum samples from patients of known H.pylori status. Serum samples were diluted 1 in 200 in phosphate buffered saline and incubated in the coated wells for 1 hour after which the wells were washed and blotted dry. Binding of specific antibody was detected using goat anti-Human IgG peroxidase, incubated for 30 minutes, then washed followed by enhanced TMB substrate (Cambridge Life Sciences). Reactions were stopped with 1 M H₂SO₄ after 15 minutes and the absorbance measured at 450 nm.

All fractions were subjected to PAGE and Western Blotting analysis to demonstrate differences in their protein contents.

(v) Page

Native gel electrophoresis was carried out using a Pharmacia Multiphor II system. A 5% gel was prepared specifically for this purpose. To 50 μl of sample 10 μl of 0.25% bromophenol blue was added and after mixing 20 μl of sample was transferred to the gel and the electrophoresis carried out (600 w for 30 minutes).

Aliquots of fractions were prepared at 1 mg/ml and 40 μl of SDS sample buffer (0.5 M HCl, pH6.8 [1.0 ml]; glycerol [0.8 ml]; 10% (w/v) SDS [1.6 ml]; 0.8 M DTT [0.4 ml]; 1% bromophenol blue [0.2 ml] and water [0.4 ml]). Samples were run in pre-prepared 10% gels (BioRad) using a BioRad Mini Protean II system. the running buffer was tris-glycine pH8.3 containing SDS (15 g Tris, 72 g glycine, 5 g SDS in 5 l distilled water). After completion of the run gels were stained wuth Coomassie Blue R-250 (0.1%) in methanol/acetic acid/water (40/10/50%) and destained in methanol/acetic acid/water (40/10/50%). Molecular weight markers used were BioRad SDS-PAGE prestained standards, broad range, consisting of Myosin 211,000; β-galactosidase 117,000; bovine serum albumin 81,000; ovalbumin 49,100; carbonic anhydrase 31,400; soybean trypsin inhibitor 26,100; lysozyme 18,900.

(vi) Western Blotting

Western Blotting analysis was used to determine which pepyides reacted only with serum from H.pylori positive patients. For each serum sample a separate gel and blot was run. SDS-PAGE gels were run as described above and transferred to nitro-cellulose membranes using a Mini Protean II Trans Blot cell (BioRad) with Towbin buffer without SDS as per the manufacturer's instructions. For the native gel transfer was achieved using the Pharmacia Multiphor II with the semi-dry blotting procedure (REF) using the discontinuous buffer system. Following transfer of proteins the nitro-cellulose membrane was washed in 20 mM Tris-HCl plus 500 mM NaCl, pH 7.5 (TBS) for 10 minutes and then blocked with 1% BSA in TBS for 1 hour. The membrane was then washed in TBS containing 0.05% Tween 20 (TTBS) and the membranes probed with serum samples diluted 1 in 60 in TTBS containing 1% BSA. Incubation was at room temperature overnight. The nitro-cellulose was then washed with TTBS and anti-Human IgG peroxidase added. Incubation for 3 hours was followed by washing in TBS after which the substrate solution (4-chloronaphthol) was added. The substrate was prepared fresh immediately before use by mixing 60 mg of 4-chloronaphthol in 20 ml of methanol with 100 ml of TBS to which 60 μl of ice-cold 30% H₂O₂ had been added immediately before the mixing process. Incubation was allowed to proceed until the substrate solution began to darken when it was replaced with fresh substrate solution. The maximum incubation time used was 30 minutes. The reaction was stopped by transferring the membrane to distilled water and washing with several changes.

The serum samples used in the assays were known to be Urea Breath Test (UBT) positive or negative and the serum status was confirmed by ELISA.

(vii) Amino Acid Sequencing

14 amino acids of the N-terminal region of 5 unique protein bands of interest were determined by solid phase analysis. SDS-PAGE and blotting were carried out as described above for Western analysis with the modification that the transfer of protein was to PDVF membrane instead of nitro-cellulose. The transfer PDVF membrane was not stained. Analysis was then completed from the solid phase using a phase sequencer (Applied Biosystems).

(viii) Testing of Patient Sera and Saliva Samples

22 patients who were being investigated for symptomatic gastric disorder, were recruited from a gastroenterology clinic (mean age 58.9 years, 12 male, 10 female). Histological examination showed that 11 were positive for H.pylori. Serum (22) and saliva (13) collected at the time of examination were tested by ELISA. Dot blot analysis of saliva was conducted on all 22 patients.

(ix) ELISA Testing

(a) Coating of ELISA Microtitre Plates with Purified Antigen

The selected, purified, antigen containing fractions off the Superose 6 column were pooled and this extract diluted to 1-2 μg protein/ml in 18.5 mM Tris-HCl plus 1 M NaCl, pH7.5. Aliquots (100 μl) of the latter were employed to coat (16 h at ambient temperature) ELISA microtitre plate wells (Nunc Maxisorb). After coating, the wells were washed three times with 5 mM phosphate buffer, containing 0.15 M NaCl and 0.01% (w/v) Thiomersal, pH7.2 (350 μl per well) and the wells were then blocked (90 min in distilled water at ambient temperature) using 1% (w/v) Byco A in distilled water (350 μl per well). After two subsequent washes (previous wash buffer), the plates were either used immediately or were dried (16 h at 37° C.) and sealed thus.

(b) Testing of Serum Samples

Sera to be tested were diluted 1 in 200 with 50 mM phosphate buffer containing 0.07% (u/v) Tween 80, 0.16& (w/v) Bromophenol Blue, 6.25% (w/v) Gelatin, 0.14 M NaCl, 0.01% (w/v) N-methylisothiazolon/HCl and 0.1% (w/v) Oxyprion, pH7.2. Aliquots (100 μl) were added to appropriate wells of an antigen coated microtitre plate (see (a) above), incubated for 45 min at ambient temperature and the wells then washed 5 times with 10 mM Tris-HCl containing 0.15 M NaCl, 0.05% (u/v) Tween 80, 0.001% (w/v) N-methylisothiazolon/HCl and 0.01% (w/v) Oxyprion, pH7.8 (350 μl per well). Binding of specific antibody was detected using rabbit anti-human IgG peroxidase conjugate (100 μl per well) suitably diluted (in 20 mM phosphate, 150 mM NaCl, 0.01% (w/v) Thiomersal, 0.1% (w/v) BSA fraction v and 0.05% (w/v) 8-anilino-1-napthalene sulphonic acid, pH7.2), with a 15 min incubation at ambient temperature. Following 5 subsequent washes (as before), TMB substrate was employed for colour development (100 μl), with the reactions stopped after 15 min at ambient temperature by the addition of 50 μl of 25% (u/v) phosphoric acid per well and the absorbance of each assay well recorded at 450 nm.

(c) Testing of Saliva Samples

saliva to be tested were diluted with 1 part Omnisal YG buffer (pH7.2, phosphate based buffer) and aliquots (100 μl) added to appropriate wells of an antigen-coated microtitre plate (see (a) above). After 30 min incubation at ambient temperature, the wells were washed 5 times (with buffer, as for serum samples) and the binding of specific antibody then detected by a Biotin-Avidin coupled assay at ambient temperature. Briefly Rb anti-human IgG Biotin (suitably diluted in 5 mM phosphate, 0.15 M NaCl, 0.05% (u/v)-Tween 80, 2.5% (w/v) Anoronthy, 1% (u/v) heat inactivated normal rabbit serum, 0.01% (w/v) Thiomersal and 2.5% (w/v) Gelatin, pH7.5) was added to each well (100 μl) and incubated for 30 min. Following 5 subsequent washes (as before), Avidin-Peroxidase conjugate (suitably diluted in 5 mM phosphate, 0.15 N NaCl, 2% (u/v) heat inactivated normal rabbit serum, 0.01% (w/v) Thiomersal and 0.05% (w/v) 8-amino-napthalene-1-sulphonic acid, pH7.2) was added to appropriate wells (100 μl), incubated for 15 min and then the wells washed 5 times as before. For colour development, TMB substrate was employed (100 μl per well), with the reactions stopped after 15 min by the addition of 50 μl of 25% (u/v) phosphoric acid per well and the absorbance of each assay recorded at 450 nm.

(x) Dot Blot Testing

To test individual fractions and pooled fractions against a single serum sample the materials to be tested were spotted onto sheets of nitrocellulose and then processed as per the Western Blot procedure detailed above. Essentially fractions or pools of the fractions containing 1 mg/ml of protein were prepared. A sheet of nitrocelulose (BioRad 8.4×7 cm cat. No. 162-0145) was marked into 24 small squares using a pencil and ruler taking care not to transfer any protein to the nitrocellulose.

Aliquots (2 μl) of each fraction or pool, containing 2 μg of protein, were carefully spotted out onto the nitrocellulose membrane within each marked square (in triplicate). The spots were allowed to dry under atmospheric conditions prior to incubation, for 1 hr at room temperature of each membrane in blocking solution (1% w/v BSA in 20 mM Tris-HCl, 500 mM NaCl pH7.5). The blocking solution was subsequently decanted, the membranes washed three times in Tween-Tris buffered saline (20 mM Tris-HCl, 500 mM NaCl 0.05% v/v Tween-20, pH7.5) and then each membrane was incubated at room temperature overnight with one of three human serum types (diluted 1:60 v/v in 1% BSA in Tween-tris buffered saline) that had been identified by HELISAL ELISA (Cortecs) test and confirmed by clinical tests as H. pylori positive, borderline or negative. Following the overnight incubation, membranes were washed twice in Tween-Tris buffered saline and then incubated for 3 hr at room temperature in conjugate solution (1:500 v/v dilution of rabbit anti-human IgG-horseradish peroxidase conjugate [Dako Cat. No. P-406] in 1% BSA in Tween-Tris buffered saline). Membranes were subsequently washed twice in Tween-Tris buffered saline, once in Tris buffered saline (20 mM Tris, 500 mM NaCl, pH7.5) and then developed for 2 to 30 minutes in 4-chloro-1-napthol solution (60 mg in 20 ml MeOH, 100 ml Tris buffered saline and 60 μl of 30% H₂O₂). Development was stopped by washing in water.

ELISA cut off values were determined in a separate study by plotting the frequency of patients with known H.pylori status, determined by histopathology, against ELISA reactivity.

Results

The elution profile of the cell free sonicate on a Mono Q HR 5/5 column is shown in FIG. 1. Fractions containing urease activity were located at an elution volume between 12 and 18 ml.

The elution profile of the Mono Q HR 5/5 column pool on a Superose 6 column gave fractions which contained urease located at an elution volume between 10 and 19 ml.

Antigen reactive fractions were determined by an ELISAgram of the Superose 6 eluate and by Western blotting. serum from patients known to be infected with H.pylori gave different ELISAgram patterns compared with uninfected subjects. A typical profile of ELISA reactivity of the Superose 6 eluate is shown in FIG. 2. An antigen preparation which gave maximum differentiation between infected and uninfected subjects was chosen for subsequent development of a diagnostic assay. The antigen fraction was chosen to the right of the main urease peak although some urease presence was detected.

Native PAGE of the reactive fraction demonstrated 16 detectable protein bands from the two strains studied with a molecular weight range of between 700 and 40 kDa (Table 1, FIG. 3).

TABLE 1
Native PAGE of reactive fraction from Superose
6 column. Molecular weights of protein bands
detected.

| | | MOLECULAR |
|---|---|---|
| BAND NO | n¹ | WEIGHT (kDax10³) |
| 1 | 9 | 665 ± 42 |
| 2 | 10 | 479 ± 32 |
| 3 | 10 | 348 ± 29 |
| 4 | 10 | 313 ± 27 |
| 5 | 8 | 246 ± 16 |
| 6 | 10 | 211 ± 20 |
| 7 | 7 | 156 ± 25 |
| 8 | 10 | 127 ± 16 |
| 9 | 10 | 109 ± 15 |
| 10 | 9 | 92 ± 7 |
| 11 | 7 | 77 ± 3 |
| 12 | 8 | 70 ± 3 |
| 13 | 9 | 63 ± 3 |
| 14 | 7 | 57 ± 3 |
| 15 | 6 | 50 ± 5 |
| 16 | 5 | 44 ± 3 |

¹Number of gel runs in which bands were observed (total 10)
Values presented are means ± SEM

Immunoblot analysis demonstrated that 9 of these protein bands were immunoreactive (Table 2, FIG. 4).

TABLE 2
Molecular weights of protein bands detected by
western blot analysis of the native PAGE
reactive fraction from the Superose 6 column

| | | MOLECULAR WEIGHT |
|---|---|---|
| IMMUNOBLOT BAND | n¹ | (kDax10³) |
| a² | 26 | 699 ± 55 |
| b | 19 | 480 ± 37 |
| c^{2,3} | 17/26 | 239 ± 17 to 326 ± 24.9 |
| d | 11 | 156 ± 25 |
| e | 12 | 109 ± 8 |
| f | 9 | 92 ± 5 |
| g | 13 | 74 ± 3 |
| h | 9 | 62 ± 5 |
| i | 11 | 36 ± 7 |

¹Number of blot analyses in which the band was observed (total 26)
²This band demonstrated urease activity
³This heavily stained region usually occurred as a single indistinguishable area (molecular weight range 240-330 kDa)
Values presented are means ± SEM

SDS-PAGE analysis of the 5 major regions observed on the native PAGE resulted in the detection of between 7 and 9 subcomponents for each region (Table 3).

TABLE 3
subunit analysis of the 5 major regions observed
on the native PAGE

| | | ESTIMATED | PRINCIPAL |
|---|---|---|---|
| MAJOR | | MOLECULAR WEIGHT | SUBUNITS |
| REGION | n¹ | RANGE (kDax10³) | (kDax10³) |
| (i) | 9 | 664 ± 42 | i-1 101 ± 8.7 |
| | | | i-2 91 ± 7.8 |
| | | | i-3 79 ± 3.8 |
| | | | i-4 72 ± 3.5 |
| | | | i-5 63 ± 6.7* |
| | | | i-6 61 ± 1.2 |
| | | | i-7 53 ± 3.8 |
| (ii) | 10 | 479 ± 32 | ii-1 109 |
| | | | ii-2 96 |
| | | | ii-3 90.5 |
| | | | ii-4 79 ± 2.3 |
| | | | ii-5 69 ± 4* |
| | | | ii-6 60 ± 2.6** |
| | | | ii-7 56 ± 1.8 |
| (iii) | 8-10 | Range 246 ± 16 to | iii-1 90 ± 4.3 |
| | | 348 ± 39 | iii-2 86 ± 3.5 |
| | | | iii-3 75 ± 2 |
| | | | iii-4 64 ± 5.1* |
| | | | iii-5 32 ± 3.8 |
| | | | iii-6 48 ± 4.5 |
| | | | iii-7 29 ± 5.0 |
| | | | iii-8 14 ± 2.5 |
| | | | iii-9 10 ± 4 |
| (iv) | 10-8 | Range 211 ± 20 to | iv-1 82 ± 3.6 |
| | | 246 ± 16 | iv-2 73 ± 2.1 |
| | | | iv-3 67 ± 2 |
| | | | iv-4 58 ± 1.2 |
| | | | iv-5 48 ± 3.3 |
| | | | iv-6 34 ± 2.6 |
| | | | iv-7 17 ± 2.9 |
| (v) | 10-7 | Range 109 ± 15 to | v-1 86 ± 4.1 |
| | | 156 ± 25 | v-2 75 ± 1.7 |
| | | | v-3 78 ± 1.7 |
| | | | v-4 65 ± 1.4 |
| | | | v-5 62 ± 3 |
| | | | v-6 54 ± 1.2 |
| | | | v-7 52 ± 1.2 |
| | | | v-8 46 ± 2.1 |
| | | | v-9 43 ± 1.7 |

¹Number of gel runs in which bands were observed
*urease positive
**strong blot
Values presented are means ± SEM

SDS PAGE analysis of the reactive fraction demonstrated 32 detectable sub-unit components 18 of which were immunoreactive with positive serum (Table 4, FIG. 5).

TABLE 4

| | | MOLECULAR WEIGHT |
|---|---|---|
| IMMUNOBLOT BAND | n¹ | (kDax10³) |
| a | 4 | 112 ± 5.4 |
| b | 5 | 100 ± 4.8 |
| c | 26 | 86.2 ± 4.3 |
| d | 24 | 78.1 ± 3.6 |
| e | 17 | 71.2 ± 2.9 |
| f₁ | 29 | 65.4 ± 3.1 |
| f₂ | 26 | 62 ± 3.3 |
| g | 14 | 57.1 ± 2.0 |
| h | 27 | 52.2 ± 2.3 |
| i₁ | 27 | 47.6 ± 1.9 |
| i₂ | 20 | 43.9 ± 2.2 |
| j | 12 | 37.7 ± 2.1 |
| k₁ | 16 | 34.1 ± 1.2 |
| k₂ | 5 | 31.6 ± 1.1 |
| L₁ | 22 | 27.5 ± 1.6 |
| L₂ | 13 | 23.2 ± 1.5 |
| m | 12 | 15.2 ± 1.5 |
| n | 12 | 13.2 ± 1.2 |

Molecular weights of subunit components detected by Western blot analysis of the SDS PAGE of the reactive fraction from the Superose 6 column Values present are means ± standard deviation. n¹ the number of analysis in which the band was detected out of 29 runs

Careful examination of the gel analysis resulted in the identification of 10 subunit components which appeared to be unique. Six of these were major bands (Table 5). N-terminal amino acid sequencing of 5 of the proteins showed that 1 corresponded to the antigen disclosed in WO-A-9625430 while 2 others were novel (no corresponding sequences described in the data bank). while the remaining 2 showed exact correspondence with known N-terminal sequences (Table 6).

TABLE 5
Unique subunit components identified in the
reactive fraction by gel analysis

| ESTIMATED MOLECULAR WEIGHT | |
|---|---|
| kDa | MAJOR COMPONENT |
| 86 | YES |
| 78 | YES |
| 72 | YES |
| 62-65 | YES |
| 57 | NO |
| 52 | YES |
| 44-48 | NO |
| 38 | NO |
| 23-27 | NO |
| 13 | YES |

TABLE 6
N-Terminal amino acid sequences of 5 proteins
identified in the reactive antigen fraction

| APPROXIMATE | | |
|---|---|---|
| MOLECULAR WEIGHT | SEQUENCE | COMMENT |
| 52 | Met-Val-Thr-Leu- | DISCLOSED IN |
| | Ile-Asn-Asn-Glu- | WO-A-962543q0 |
| | Asp-Asp | |
| 53 | Met-Asp-Leu-?- | NOVEL |
| | Val-Leu-Gly-Ile- | |
| | Asn-Thr-Ala | |
| 43 | ?-?-Gly-Lys-Ala- | NOVEL |
| | Pro-Asp-Phe-Lys- | |
| | Pro-Ala | |
| 57 | Ala-Lys-Glu-Iso- | HEAT SHOCK |
| | Lys-Phe-Ser-Asp | PROTEIN B |
| 62-65 | Met-Lys-Lys-Ile- | Urease B-SUBUNIT |
| | Ser-Arg-Lys-Glu | |

The usefulness of the reactive fraction, which contains the novel antigens, in a diagnostic ELISA was tested. Cut-offs were determined to be 0.7 ELISA units for saliva and 2.5 ELISA units for serum (FIG. 6).

Table 7 shows the performance of the serum and salivary ELISA and dot blot assays against the detection of H.pylori infection by histology. The results show that both serum and saliva are highly sensitive with excellent positive and negative predictive values. Salivary dot blot analysis gave acceptable performance measure although not as high as saliva or serum ELISA.

TABLE 7
Comparison of salivary and serum antibody
determined by ELISA or dot blot against
histology (antral biopsy)

| | | | | | POSITIVE | NEGATIVE |
|---|---|---|---|---|---|---|
| | | | | | PREDICTIVE | PREDICTIVE |
| TEST | n | SENSITIVITY | SPECIFICLY | ACCURACY | VALUE | VALUE |
| SALIVA | 22 | 90.0 | 81.8 | 86.4 | 83.3 | 90.0 |
| DOT | | | | | | |
| BLOT | | | | | | |
| SALIVA | 13 | 100.0 | 85.7 | 92.3 | 85.7 | 100.0 |
| ELISA | | | | | | |
| SERUM | 22 | 100.0 | 90.9 | 95.4 | 91.6 | 100.0 |
| ELISA | | | | | | |

### EXAMPLE 2

(i)

Cultures of H. pylori were grown under appropriate conditions and the cells harvested into phosphate buffered saline. This was followed by repeated centrifugation to remove cell debris and other contaminants, for example agar, and fresh PBS was added three times to yield a washed cell pellet;

(b) The washed cells were resuspended in 0.1 M TRIS-HCl buffer pH 7.2 to be used in the ion exchange chromatography step. The cell suspension was then subjected to sonication (6μ for 30 seconds, 60 seconds off, repeated 25 times for a 10 ml sample containing cells from 100 agar plates) of sufficient intensity and duration to ensure disruption of the cells;

(c) The suspension was then centrifuged to remove cell debris and the supernatant, containing soluble cell proteins, was obtained;

(d) The solution from step (c) was then subjected to fractionation by ion-exchange chromatography using a strong anion exchange resin such as MonoQ® or Q-Sepharose® (Pharmacia), using a gradient elution based on increasing the sodium chloride concentration of the elution buffer from 0 to 1.0 M in a predetermined manner. The fractions were then assayed for the presence of urease;

(e) The urease containing fractions were then pooled and were subjected to gel permeation chromatography using a resin with a cut-off range of 5×10³−5×10⁶ Da for globular protein;

(f) The appropriate peak was selected by:

(i) carrying out a urease assay of all the fractions and identifying the protein peak containing the urease activity;

(ii) analysing all the fractions shown to be urease positive and those protein peaks immediately adjacent to the urease peak but of lower (apparent) molecular weight by western blot analysis of the native protein and of fragments thereof produced by denaturing (SDS) treatments using IgG from a pool prepared from human serum collected from H.pylori positive individuals; and

(iii) selecting those bands of protein which following electrophoretic separation in the above manner were shown to react with the human IgG from a positive pool of serum but not with a similar serum pool prepared from H.pylori negative serum.

Each band thus identified was subjected to N-terminal amino acid analysis and the sequences were compared with sequences for known proteins from available computer databases.

In a particular embodiment, this invention provides a kit for detection or diagnosis of H. pylori in a sample from a patient. The kit contains at least one or more antigens or antigenic fragments according to this invention, along with the means to detect binding between the antigens or fragments and antibodies which specifically bind such antigens or fragments. Selection of suitable means for detecting antigen-antibody binding is easily within the skill of the ordinary worker in this art, and include primary and/or secondary labeled antibodies to IgG from humans or other mammals, and/or other known materials for sandwich assays, ELISA assays, competitive immunoassays, and other well known immunometric assay formats.

1. A Helicobacter pylori antigenic protein having the following characteristics:
(a) a molecular weight of about 53 kDa, as determined under denaturing and reducing conditions, and having the following N-terminal amino acid:
Met Asp Leu Xaa Val Leu Gly Ile Asn Thr Ala (SEQ. ID. NO 1);
(b) a molecular weight of about 43 kDa, as determined under denaturing and reducing conditions, and having an N-terminal amino acid sequence selected from the group consisting of:
Met Arg Val Pro Lys Lys Gly Phe Ala Ile Leu Ser Lys (SEQ. ID. NO 2); and
Met Arg Val Pro Ser Lys Gly Phe Ala Ile Leu Ser Lys (SEQ. ID. NO 3);
(c) a molecular weight of about 43 kDa, as determined under denaturing and reducing conditions, and having the following N-terminal amino acid sequence:
Xaa Xaa Gly Lys Ala Pro Asp Phe Lys Pro Ala (SEQ. ID. NO 4);
(d) a molecular weight of about 54 kDa, as determined under denaturing and reducing conditions, and having an N-terminal amino acid sequence selected from the group consisting of:

| | |
|---|---|
| Met Leu Lys Ile Lys Leu Glu Ile | (SEQ. ID. NO 5); |
| Met Leu Lys Ile Lys Leu Ser Ile | (SEQ. ID. NO 6); |
| Met Leu Lys Ile Val Leu Glu Ile | (SEQ. ID. NO 7); |
| Met Leu Lys Ile Val Leu Ser Ile | (SEQ. ID. NO 8); |
| Met Leu Lys Ile Ser Leu Glu Ile | (SEQ. ID. NO 9); |
| Met Leu Lys Ile Ser Leu Ser Ile | (SEQ. ID. NO 10); |
| Met Leu Lys Glu Lys Leu Glu Ile | (SEQ. ID. NO 11); |
| Met Leu Lys Glu Lys Leu Ser Ile | (SEQ. ID. NO 12); |
| Met Leu Lys Glu Val Leu Glu Ile | (SEQ. ID. NO 13); |
| Met Leu Lys Glu Val Leu Ser Ile | (SEQ. ID. NO 14); |
| Met Leu Lys Glu Ser Leu Glu Ile | (SEQ. ID. NO 15); |
| Met Leu Lys Glu Ser Leu Ser Ile | (SEQ. ID. NO 16); |
| Met Leu Val Ile Lys Leu Glu Ile | (SEQ. ID. NO 17); |
| Met Leu Val Ile Lys Leu Ser Ile | (SEQ. ID. NO 18); |
| Met Leu Val Ile Val Leu Glu Ile | (SEQ. ID. NO 19); |
| Met Leu Val Ile Val Leu Ser Ile | (SEQ. ID. NO 20); |
| Met Leu Val Ile Ser Leu Glu Ile | (SEQ. ID. NO 21); |
| Met Leu Val Ile Ser Leu Ser Ile | (SEQ. ID. NO 22); |
| Met Leu Val Glu Lys Leu Glu Ile | (SEQ. ID. NO 23); |
| Met Leu Val Glu Lys Leu Ser Ile | (SEQ. ID. NO 24); |
| Met Leu Val Glu Val Leu Glu Ile | (SEQ. ID. NO 25); |
| Met Leu Val Glu Val Leu Ser Ile | (SEQ. ID. NO 26); |
| Met Leu Val Glu Ser Leu Glu Ile | (SEQ. ID. NO 27); |
| | and |
| Met Leu Val Glu Ser Leu Ser Ile | (SEQ. ID. NO 28); |

(e) a molecular weight of about 370 kDa, as determined under native (non-denaturing) conditions, and having an N-terminal amino acid sequence selected from the group consisting of:

| | |
|---|---|
| Met Leu Thr Ile Xaa Leu Glu Val | (SEQ. ID. NO 29); |
| Met Leu Thr Ile Xaa Leu Glu Glu | (SEQ. ID. NO 30); |
| Lys Leu Thr Ile Xaa Leu Glu Val | (SEQ. ID. NO 31); |
| | and |
| Lys Leu Thr Ile Xaa Leu Glu Glu | (SEQ. ID. NO 32); |

(f) a molecular weight of about 140 kDa, as determined under native (non-denaturing) conditions, and having an N-terminal amino acid sequence selected from the group consisting of:
Met Tyr Ile Pro Tyr Val Ile Glu (SEQ. ID. NO 33);
(g) a molecular weight of about 90 kDa, as determined under native (non-denaturing) conditions and having an N-terminal amino acid sequence selected from the group consisting of:

| | |
|---|---|
| Met Asn Leu Asp Cys Leu Gln Val | (SEQ. ID. NO 34); |
| | and |
| Met Asn Leu Asp Ser Leu Gln Val | (SEQ. ID. NO 35); |

(h) a molecular weight of about 15.9 to 16.9 kDa, as determined under denaturing and reducing conditions, and having the following N-terminal amino acid sequence:
Gly Lys Ile Gly Ile Phe Phe Gly Thr Asp Ser Gly Asn Ala Glu Ala Ile Ala Glu Lys (SEQ. ID. NO. 36;
(i) a molecular weight of about 334 kDa, as determined under native (non-denaturing) conditions, and having the following N-terminal amino acid sequence:
Met Leu Val Thr Lys Leu Ala Pro Asp Phe Leu Ala Pro Xaa Val (SEQ. ID. NO. 37).

2. An antigenic fragment of a protein defined in claim 1.

3. The antigenic fragment recited in claim 2 having the sequence:

| | | |
|---|---|---|
| (i) | Met Asp Leu Xaa Val Leu Gly Ile Asn Thr Ala | (SEQ. ID. NO. 1); |
| (ii) | Met Arg Val Pro Lys Lys Gly Phe Ala Ile Leu Ser Lys | (SEQ. ID. NO. 2); |
| (iii) | Met Arg Val Pro Ser Lys Gly Phe Ala Ile Leu Ser Lys | (SEQ. ID. NO. 3); |
| (iv) | Xaa Xaa Gly Lys Ala Pro Asp Phe Lys Pro Ala | (SEQ. ID. NO. 4); |
| (v) | Met Leu Lys Ile Lys Leu Glu Ile | (SEQ. ID. NO. 5); |
| (vi) | Met Leu Lys Ile Lys Leu Ser Ile | (SEQ. ID. NO. 6); |
| (vii) | Met Leu Lys Ile Val Leu Glu Ile | (SEQ. ID. NO. 7); |
| (viii) | Met Leu Lys Ile Val Leu Ser Ile | (SEQ. ID. NO. 8); |
| (ix) | Met Leu Lys Ile Ser Leu Glu Ile | (SEQ. ID. NO. 9); |
| (x) | Met Leu Lys Ile Ser Leu Ser Ile | (SEQ. ID. NO. 10); |
| (xi) | Met Leu Lys Glu Lys Leu Glu Ile | (SEQ. ID. NO. 11); |
| (xii) | Met Leu Lys Glu Lys Leu Ser Ile | (SEQ. ID. NO. 12); |
| (xiii) | Met Leu Lys Glu Val Leu Glu Ile | (SEQ. ID. NO. 13); |
| (xiv) | Met Leu Lys Glu Val Leu Ser Ile | (SEQ. ID. NO. 14); |
| (xv) | Met Leu Lys Glu Ser Leu Glu Ile | (SEQ. ID. NO. 15); |
| (xvi) | Met Leu Lys Glu Ser Leu Ser Ile | (SEQ. ID. NO. 16); |
| (xvii) | Met Leu Val Ile Lys Leu Glu Ile | (SEQ. ID. NO. 17); |
| (xviii) | Met Leu Val Ile Lys Leu Ser Ile | (SEQ. ID. NO. 18); |
| (xix) | Met Leu Val Ile Val Leu Glu Ile | (SEQ. ID. NO. 19); |
| (xx) | Met Leu Val Ile Val Leu Ser Ile | (SEQ. ID. NO. 20); |
| (xxi) | Met Leu Val Ile Ser Leu Glu Ile | (SEQ. ID. NO. 21); |
| (xxii) | Met Leu Val Ile Ser Leu Ser Ile | (SEQ. ID. NO. 22); |
| (xxiii) | Met Leu Val Glu Lys Leu Glu Ile | (SEQ. ID. NO. 23); |
| (xxiv) | Met Leu Val Glu Lys Leu Ser Ile | (SEQ. ID. NO. 24); |
| (xxv) | Met Leu Val Glu Val Leu Glu Ile | (SEQ. ID. NO. 25); |
| (xxvi) | Met Leu Val Glu Val Leu Ser Ile | (SEQ. ID. NO. 26); |
| (xxvii) | Met Leu Val Glu Ser Leu Glu Ile | (SEQ. ID. NO. 27); |
| (xxviii) | Met Leu Val Glu Ser Leu Ser Ile | (SEQ. ID. NO. 28); |
| (xxix) | Met Leu Thr Ile Xaa Leu Glu Val | (SEQ. ID. NO. 29); |
| (xxx) | Met Leu Thr Ile Xaa Leu Glu Glu | (SEQ. ID. NO. 30); |
| (xxxi) | Lys Leu Thr Ile Xaa Leu Glu Val | (SEQ. ID. NO. 31); |
| (xxxii) | Lys Leu Thr Ile Xaa Leu Glu Glu | (SEQ. ID. NO. 32); |
| (xxxiii) | Met Tyr Ile Pro Tyr Val Ile Glu | (SEQ. ID. NO. 33); |
| (xxxiv) | Met Asn Leu Asp Cys Leu Gln Val | (SEQ. ID. NO. 34); |
| (xxxv) | Met Asn Leu Asp Ser Leu Gln Val | (SEQ. ID. NO. 35); |
| (xxxvi) | Gly Lys Ile Gly Ile Phe Phe Gly Thr Asp Ser Gly Asn Ala Glu Ala Ile | SEQ. ID. NO. 36); |
| | Ala Glu Lys | |
| (xxxvii) | Met Leu Val Thr Lys Leu Ala Pro Asp Phe Leu Ala Pro Xaa Val | (SEQ. ID. NO. 37); or |
| (xxxviii) | Met Phe Thr Leu Arg Glu Leu Pro Phe Ala Lys Asp Ser Asn Gly Asp | (SEQ. ID. NO. 38). |
| | Phe Leu Ser Pro | |

4. An antigen composition comprising one or more of the proteins defined in claim 1.

5. An antigen composition comprising the antigenic fragment defined in claim 2.

6. An antigen composition comprising one or more of the antigenic fragments defined in claim 3.

7. The antigen composition recited in claim 4 further comprising one or more other H.pylori antigens and/or fragments thereof.

8. The antigen composition recited in claim 6 further comprising one or more other H. pylori antigens and/or fragments thereof.

9. Superoxide dismutase enzyme from H. pylori having the following N-terminal amino acid sequence:
Met Phe Thr Leu Arg Glu Leu Pro Phe Ala Lys Asp Ser Asn Gly Asp Phe Leu Ser Pro (SEQ. ID. NO. 38) for use in the detection or diagnosis of H. pylori.

10. A method of detecting or diagnosing H. pylori comprising:
(a) bringing at least one or more of the antigenic proteins defined in claim 1 into contact with a sample to be tested; and
(b) detecting the presence of antibodies to H. pylori.

11. The method recited in claim 1 0 wherein the sample is a sample of saliva.

12. A method of detecting or diagnosing H. pylori comprising:
(a) bringing at least one or more of the antigenic fragments as defined in claim 3 into contact with a sample to be tested; and
(b) detecting the presence of antibodies to H. pylori.

13. The method recited in claim 10 wherein the sample is a sample of blood.

14. A method of detecting or diagnosing H. pylori comprising:
(a) bringing the superoxide dismutase defined in claim 9 into contact with a sample to be tested; and
(b) detecting the presence of antibodies to H. pylori.

15. The method recited in claim 14 wherein the sample is a sample of blood.

16. A kit for use in the detection or diagnosis of H. pylori comprising one or more of the proteins defined in claim 1.

17. A kit for use in the detection or diagnosis of H. pylori comprising the antigen composition defined in claim 5.

18. A composition capable of eliciting an immune response in a subject comprising the antigenic fragment defined in claim 2.

20. The composition recited in claim 18 further comprising one or more adjuvants.

21. A composition capable of eliciting an immune response in a subject comprising the antigen composition defined in claim 5.

22. The composition recited in claim 21 further comprising one or more adjuvants.

23. A method for the treatment of prophylaxis of H. pylori infection in a subject comprising the step of administering to the subject an effective amount of the antigenic fragment defined in claim 2.

24. A method for the treatment of prophylaxis of H. pylori infection in a subject comprising the step of administering to the subject an effective amount of the antigenic composition defined in claim 6.

25. A method for the treatment of prophylaxis of H. pylori infection in a subject comprising the step of administering to the subject an effective amount of the superoxide dismutase enzyme defined in claim 9.

26. The method recited in claim 23 wherein the antigenic fragment is administered in the form of a vaccine.

27. The method recited in claim 25 wherein the superoxide dismutase enzyme is administered in the form of a vaccine.
